(19) United States
(12) Patent Application Publication
(10) Pub. No.: US 2012/0114592 A1
(43) Pub. Date: May 10, 2012

(54) Hemostatic Foams

(75) Inventors: Johan Zuidema, Aduard (NL); Jacob Richard de Vries, Groningen (NL); Jhacintha Sarikadevie Jankie, Hoogezand (NL); Rudolf Robert Maria Bos, Groningen (NL); Susan Henrieke Visscher, Middelstum (NL)

(73) Assignee: Polyganics B.V., Groningen (NL)

(21) Appl. No.: 13/322,671

(22) PCT Filed: May 27, 2010
(86) PCT No.: PCT/NL2010/050321

§ 371 Date: Jan. 25, 2012

(30) Foreign Application Priority Data
May 27, 2009 (NL) 2002931

(51) Int. Cl. A61L 26/00; C08L 75/08; C08L 75/06; C08L 75/04; C08J 9/00

(52) U.S. Cl. 424/78.06; 521/137

(57) ABSTRACT

Hemostatic foams Abstract The invention is directed to hemostatic foams and to the preparation of such foams. In a first aspect, the present invention provides a biodegradable hemostatic foam comprising a polymer blend of a water-soluble polymer and a phase-separated polyurethane comprising an amorphous segment and a crystalline segment, wherein at least said amorphous segment comprises a hydrophilic segment.

The invention is directed to hemostatic foams and to the preparation of such foams.

Hemostasis is a process in the body of humans and animals that causes the bleeding of wounds, e.g. damaged blood vessels, to stop. Hemostasis is of fundamental importance for the success of surgical operations as well as subsequent wound healing. A hemostatic foam is intended to produce hemostasis by accelerating the clotting process of blood by applying the foam locally to a bleeding surface.

All commercially available hemostatic foams are currently based on materials derived from animals (e.g. collagen and gelatin) or plants (e.g. cellulose). A disadvantage of these foams is that using such natural materials increases the risk of transfer of diseases, such as for example bovine spongiform encephalopathy (mad cow disease). A further disadvantage of commercially available hemostatic foams based on collagen and gelatin is that they loose their compression strength once saturated with blood, which makes it almost impossible to manipulate the foams after application.

(Fully) synthetic hemostatic foams would not suffer from these drawbacks. Because the materials used to produce such foams are not derived from animal products, there is no risk of transmission of pathogens using such foams. A further advantage of synthetic foams lies in its production process, which is simple and easy to control.

WO-A-90/13320 describes a hemostatic sponge based on a natural material, such as gelatin or cellulose, which sponge comprises thrombin and a thrombin-stabilizing agent. This hemostatic sponge can be prepared by injecting small amounts of thrombin solution comprising a stabilizing agent into a sponge based on a natural material. The structure of the natural material is not changed during injection. The stabilizing agent may be a polyvalent alcohol such as polyethylene glycol. Disadvantage of this sponge is its lack of elastic properties and dependence on thrombin present in the foam for its hemostatic properties.

From WO-A-99/64491 it is known that polyurethanes having a phase-separated morphology exhibit good physical and mechanical properties and are generally easy to process.

WO-A-2004/062704 describes a hydrophilic biodegradable foam, comprising a biodegradable synthetic phase-separated polymer including an amorphous segment and a crystalline segment, wherein said amorphous segment comprises a hydrophilic segment. The presence of a hydrophilic segment or group in the amorphous phase of the polymer provides the foam with characteristics such as the capacity to absorb aqueous liquids and being readily biodegradable. The foam may be used for controlling bleeding (hemostatic sponge), for wound closure, for the prevention of tissue adhesion and/or for support tissue regeneration and is suitable for packing antrums or other cavities of the human or animal body. The foam has the advantage that it does not have to be mechanically removed after being applied to an antrum, such as the nasal cavity, since it degrades over time.

Disadvantages of the foams known from the WO-A-99/64491 and WO-A-2004/062704 are that these foams are unable to absorb aqueous liquids at high speeds. Therefore, these foams can not be suitably used as hemostatic foams for bleeding, in particular for heavy bleeding, because they are unable to absorb the excess amount of blood released from bleeding wounds, e.g. surgical wounds.

It is an object of the present invention to provide a biodegradable synthetic hemostatic foam that rapidly absorbs blood while retaining at least part of its compression strength, in particular essentially retaining its compression strength during the (critical) healing period of the tissue. It is also an object of the present invention to provide a method for preparing such a foam.

The inventors found that this object can be met by providing a foam comprising a polymer blend of a water-soluble polymer and a specific polyurethane.

In a first aspect, the present invention provides a biodegradable hemostatic foam comprising a polymer blend of a water-soluble polymer and a phase-separated polyurethane, which polyurethane comprises an amorphous segment and a crystalline segment, wherein at least said amorphous segment comprises a hydrophilic segment.

It was surprisingly found that a foam according to the present invention, when applied to a bleeding surface, increases the coagulation speed of the blood. Without wishing to be bound by theory, this increase in coagulation speed is believed to be caused by the presence of the water-soluble polymer in the foam, which polymer provides the foam with a hydrophilic nature and the ability to rapidly absorb water. It is believed, again without wishing to be bound by theory, that the hydrophilic hemostatic foam of the invention, when applied to a bleeding surface, manages to locally increase the concentration of platelets and other hemostasis stimulating compounds by absorbing water from the blood. The increased concentration then promotes coagulation of the blood and contributes to quick and efficient hemostasis. Foams of the present invention can absorb blood at the same rate and equally effective as present commercially available hemostatic foams based on collagen and gelatin.

It was further found that with a foam of the present invention it is possible to retain most of its compression strength once saturated with blood, in particular to essentially retain the compression strength once saturated with blood, which makes them easy to manipulate after application.

The term “polymer blend” as used herein is defined as a mixture of two or more different polymers. The polymers in a polymer blend are preferably randomly distributed throughout the blend. In a polymer blend, cross-linking between the two polymers is typically avoided.

The foam of the present invention keeps its structure upon absorbing blood. The water-soluble polymer present in the polymer blend does not have to dissolve and leave the foam, but may continue to be part of the foam, while the foam retains its structure. During absorption of blood, the water soluble polymer may partly dissolve in blood and may diffuse from the polymeric matrix into the pores of the foam. However the water soluble polymer will typically not leave the foams due to the blood clot forming in the pores. After application, degradation and resorption of the polyurethane and the water soluble polymer will start. During this period the water soluble polymer may dissolve in body fluids and can be excreted from the body.

Preferably, the polymer blend in the foam according to the present invention comprises about 0.5 to 20 times as much phase-separated polyurethane as water-soluble polymer by weight. Thus the weight ratio [phase-separated polyurethane]/[water-soluble polymer] is preferably 0.5-20, more preferably 1-10, even more preferably 2-5. Most preferably, said polymer blend comprises about 3 times as much biodegradable polyurethane as water-soluble polymer by weight. When the polymer blend comprises less than 0.5 times as much polyurethane as water-soluble polymer will generally result in foams with insufficient strength to be able to manipulate after application. When on the other hand the polymer blend comprises more than 20 times as much polyurethane as water-soluble polymer, this will generally result in foams with the insufficient capacity to rapidly absorb blood.

The term “water-soluble polymer” as used herein, refers to a polymer with affinity for water. It is per definition a hydrophilic polymer and must thus comprise one or more hydrophilic groups. Due to its affinity for water, a water-soluble polymer is both able to absorb water, as well as to dissolve in water. Preferably at least 50% of the atoms in the main chain of a water-soluble polymer is either part of a hydrophilic group or connected to a hydrophilic group through 2 atoms or less. For example, in polyvinyl alcohol CH₂—CH(OH)ₙ the carbon atom in CH₂ is connected through one atom, viz. a carbon atom, to the hydrophilic hydroxyl group. Examples of hydrophilic groups are hydroxyl, ether and amine groups.

A water-soluble polymer typically comprises hydrophilic segments, i.e. segments that comprise at least one or more hydrophilic groups. A hydrophilic segment may be provided by for example polyether, polypeptide, poly(vinyl alcohol), poly(vinyl pyrrolidone) or poly(hydroxymethyl methacrylate). A hydrophilic segment is preferably derived form polyalkylene glycol, such as polyethylene glycol, polypropylene glycol, or polybutylene glycol. The preferred hydrophilic segment is a polyethylene glycol (PEG) segment.

The water-soluble polymer may be chosen from the group consisting of polyether, polypeptide, poly(vinyl alcohol), poly(vinyl pyrrolidone), poly(hydroxymethyl methacrylate), polysaccharides, polyvinylpyrrolidone and copolymers, as well as combinations thereof. Preferably, the water-soluble polymer used in the invention is polyalkylene glycol, more preferably polyethylene glycol. The water-soluble polymer may also be polyurethane. More preferably, the water-soluble polymer is selected from poly(vinyl pyrrolidone), poly(vinyl alcohol), or combinations thereof.

The polymer blend further comprises a phase-separated polyurethane comprising an amorphous segment and a crystalline segment, wherein at least said amorphous segment comprises a hydrophilic segment. Such a polyurethane is described in WO-A-2004/062704.

A hemostatic foam is said to be hemocompatible if, upon contact with blood, the hemostatic foam does not cause therapeutically detrimental alterations to the blood. Polyurethane is well known for its hemocompatible properties. The polymer blend according to the invention appears to have retained these favorable properties of polyurethane. It was found that the foams according to the invention mainly cause a passive activation of the clotting cascade and does not alter the blood composition in any noticeable way. Furthermore, the surface structure of the foam of the present invention makes it particularly hemocompatible. Without wishing to be bound by theory, it is believed that this hemocompatibility is achieved by the balance of polar sites, provided by both polyurethane and the water-soluble polymer, and non-polar sites, provided by polyurethane, at the surface of the hemostatic foam of the invention.

As described in WO-A-2004/062704, the amorphous segment of the polyurethane must comprise a hydrophilic segment. This amorphous segment, also called the amorphous phase in the art, is amorphous when applied to a bleeding surface, i.e. when wet, despite the fact that it may comprise a crystalline polyether. This means that, in the dry state, said crystalline polyether may provide the amorphous phase of the polymer with partially crystalline properties. The performance of the foam when applied to a bleeding surface determines the characteristics of the foam: when applied to a bleeding surface, the foam of the invention is comprised of an amorphous hydrophilic soft segment or phase and a crystalline hard segment or phase.

Hydrophilic groups may also be present in the hard segment of the polyurethane, but the presence of hydrophilic groups in the hard segment should not result in immediate disintegration of the foam when placed in contact with fluids. Essentially, the crystalline hard segment or phase must provide the foam with rigidity, keep the foam intact and prevent swelling of the foam when placed in contact with fluids.

The term polyurethane as used in this application is defined as a polymer comprising one or more links between either amide, urea or urethane.

The term “biodegradable” as used herein, refers to the ability of a polymer to be acted upon biochemically in general by living cells or organisms or part of these systems, including hydrolysis, and to degrade and disintegrate into chemical or biochemical products.

The term “phase-separated polyurethane” as used herein, refers to a polyurethane comprising soft (amorphous) segments, as well as hard (crystalline) segments, the phase-separated morphology being manifest when the foam prepared from such a polyurethane is applied to a bleeding surface of a human or animal body for a sufficient period of time. Also, the polyurethane placed under temperature conditions comparable to the human or animal body exhibits said phase-separated morphology.

A phase-separated polyurethane is characterised by the presence of at least two immiscible or partly miscible phases with a different morphology and different thermal state at normal environmental conditions. Within one material a soft rubbery amorphous phase and a hard crystalline phase (at a temperature above the glass transition temperature of the amorphous phase and below the melting temperature of the crystalline phase) may be present or a hard glassy amorphous phase and a hard crystalline phase (at a temperature below the glass transition temperature of the amorphous phase). Also at least two amorphous phases can be present, e.g. one hard glassy and one soft rubbery phase. Even above the melting temperature, the liquid and rubbery phases can still be immiscible. More in particular, when a polyurethane has an amorphous phase and a crystalline phase, which two phases are immiscible with each other, the polyurethane is said to be phase-separated. The presence of immiscible phases (amorphous and crystalline) may be suitably determined by the use of a e.g. (modulated) differential scanning calorimetry (DSC).

The phase separated morphology is essential for the mechanical properties of the foams. Both phases contribute to the unique properties of the material of the present invention. The soft, amorphous phase is responsible for the flexible and elastic behavior. The hard, crystalline phase is responsible for the hardness and strength of the material. The (semi) crystalline hard segments undergo intermolecular crystallization and behave as physical cross-links and knot the soft segments in a three dimensions network structure. Because of this, microphase separation may appear where the hard crystalline and the soft amorphous segments can form a cocontinuous two-phase system. Cocontinuous microstructures are characterized by having both phases interpenetrating each other in three dimensions. In a cocontinous morphology all the hard areas are connected with each other. Due to this morphology the foam has the ability to essentially maintain its compression strength upon blood absorption.

The term “amorphous” as used herein, refers to segments present in the polyurethane of the invention with at least one glass transition temperature below the temperature of the bleeding surface and may also refer to a combination of an amorphous and crystalline segment which is completely amorphous when applied to a bleeding surface. The glass transition temperature may be determined with the use of a (modulated) differential scanning calorimeter.

The term “crystalline” as used herein, refers to segments, present in the polyurethane of the invention, that are crystalline when applied to a bleeding surface, that have a melting temperature above the temperature of the bleeding surface.

A “hydrophilic segment” as used herein, refers to a segment comprising at least one, preferably at least two, more preferably at least three hydrophilic groups, which can for instance be provided by C—O—C, or ether, linkages. A hydrophilic segment may thus be provided by a polyether segment. A hydrophilic segment may also be provided by polypeptide, poly(vinyl alcohol), poly(vinylpyrrolidone) or poly(hydroxymethylmethacrylate). A hydrophilic segment is preferably derived from polyalkyleneglycol, such as polyethyleneglycol, polypropyleneglycol, or polybutyleneglycol. The preferred hydrophilic segment is a polyethyleneglycol (PEG) segment.

The term “segment” as used herein, refers to a polymeric structure of any length. In the art of polymer technology a long polymeric structure is often referred to as a block, whereas a short polymeric structure is often referred to as a segment. Both these conventional meanings are understood to be comprised in the term “segment” as used herein.

In one embodiment of the foam of the invention, the polymer blend comprises a phase-separated, biodegradable polyurethane of formula (I):

R-Q¹[-R′-Z¹-[R″-Z²-R″′-Z³]ₚ-R″-Z⁴]_{q}-R′-Q²ₙ (I)

wherein R is a polymer or copolymer selected from one or more aliphatic polyesters, polyether esters, polyethers, polyanhydrides, and/or polycarbonates, and at least one R comprises a hydrophilic segment; R′, R″ and R″′ are independently C₂-C₈ alkylene, optionally substituted with C₁-C₁₀ alkyl or C₁-C₁₀ alkyl groups substituted with protected S, N, P or O moieties and/or comprising S, N, P or O in the alkylene chain; Z¹-Z⁴ are independently amide, urea or urethane, Q¹ and Q² are independently urea, urethane, amide, carbonate, ester or anhydride, n is an integer from 5-500; and p and q are independent 0 or 1.

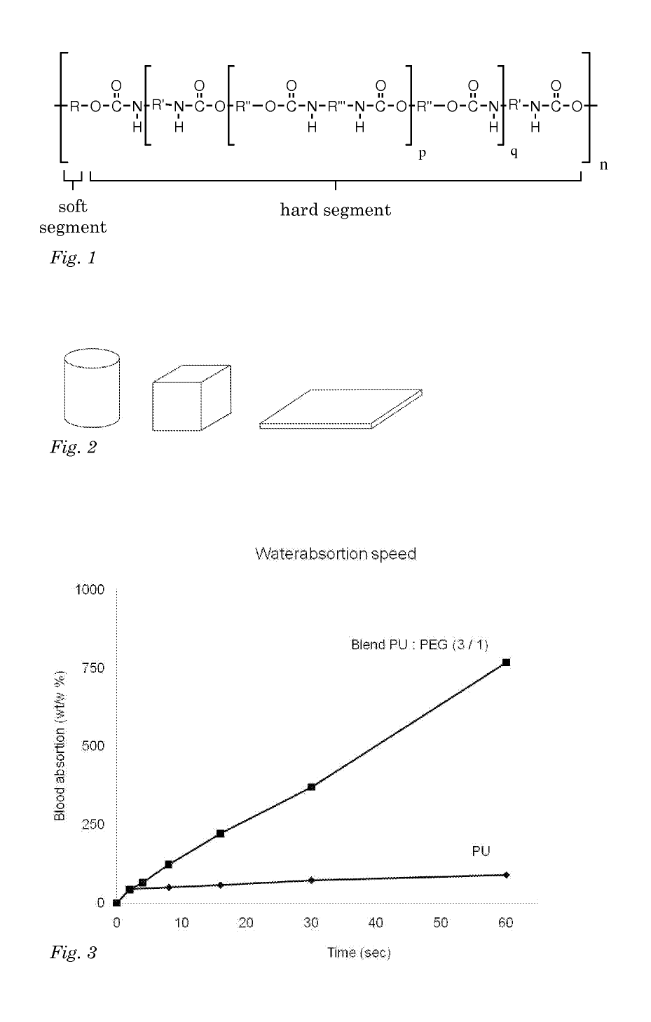

The soft segment of the polyurethane of formula (I) is generally represented by R, whereas the remainder of formula (I) generally represents the hard segment of the polyurethane. The division of the polyurethane of formula (I) in hard and soft segments is also schematically shown in FIG. 1.

Although Z¹-Z⁴ may differ from each other, Z¹-Z⁴ are preferably chosen to be the same. More preferably, Z¹-Z⁴ are all urethane moieties and the polyurethane can in such a case be represented by formula (II):

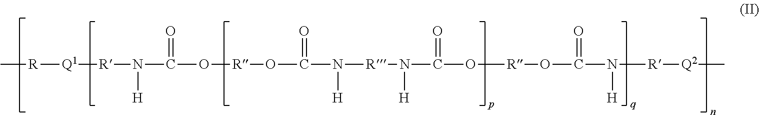

wherein Q¹, Q², R, R′, R″, R″′, p, q and n are defined as described hereinabove for formula (I).

Q¹ and Q² are chosen independently from each other from the group consisting of urea, urethane, amide, carbonate, ester and anhydride. Preferably, Q¹ and Q² are independently chosen from urethane, carbonate and ester. Although Q¹ and Q² may be chosen to be different kind of moieties, Q¹ and Q² are preferably the same.

Preferably, q=1 in formulas (I) and (II). Thus, the polyurethane has a hard segment of sufficient length to easily form crystalline domains, resulting in a phase-separated polyurethane. An even more desirable length is obtained for this purpose if both q and p equal 1.

To enhance the phase-separated nature of a polyurethane, R can be chosen as a mixture of an amorphous and a crystalline segment. For this purpose, R is preferably a mixture of at least one crystalline polyester, polyether ester or polyanhydride segment and at least one amorphous aliphatic polyester, polyether, polyanhydride and/or polycarbonate segment. This may be particularly desirable when q is chosen 0, because the urethane moiety may in such a case be too small to form crystalline domains, resulting in a mixture of both phases, wherein no phase-separation occurs.

According to the present invention, the amorphous segment is comprised in the -R- part of the polyurethane according to formula (I). The remaining part of the polymer according to formula (I), including the R′, R″ and R″′ units, represents the crystalline segment. The crystalline segment is always a hard segment, while the amorphous segment at least comprises one or more soft segments. R in formula (I) comprises the soft segments, while the remainder of formula 1 typically comprises the hard segments. The separation of the polyurethane in soft segments and hard segments is also shown in FIG. 1. The soft segments are typically amorphous in the polyurethane of the invention. The hard segments have a tendency to crystallize, but may be amorphous when not crystallized completely.

R is a polymer or copolymer selected from aliphatic polyesters, polyether esters, polyethers, polyanhydrides, polycarbonates and combinations thereof, wherein at least one hydrophilic segment is provided in at least one amorphous segment of R. Preferably, R is a polyether ester. R can for example be a polyether ester based on DL lactide and ε-caprolactone, with polyethylene glycol provided in the polyether ester as a hydrophilic segment.

R comprises a hydrophilic segment and such a hydrophilic segment can very suitably be an ether segment, such as a polyether segment derivable from such polyether compounds as polyethyleneglycol, polypropyleneglycol or polybutyleneglycol. Also, a hydrophilic segment comprised in R may be derived from polypeptide, poly(vinyl alcohol), poly(vinylpyrrolidone) or poly(hydroxymethylmethacrylate). A hydrophilic segment is preferably a polyether. Each of the groups R′, R″ and R″′ is a C₂-C₈ alkylene moiety, preferably a C₃-C₆ alkylene moiety. The alkylene moiety may be substituted with C₁-C₁₀ alkyl or C₁-C₁₀ alkyl groups substituted with protected S, N, P or O moieties and/or comprising S, N, P or O in the alkylene chain. Preferably, the alkylene moiety is unsubstituted (CₙH₂ₙ) or substituted. R′, R″ and R″′ may all be chosen to be a different alkylene moiety, but may also be the same.

Preferably, R′ is an unsubstituted C₄ alkylene (C₄H₈) or an unsubstituted C₆ alkylene (C₆H₁₂). R′ may be derived from a diisocyanate of the formula O═C═N—R′—N═C═O, such as alkanediisocyanate, preferably 1,4-butanediisocyanate (BDI) or 1,6-hexanediisocyanate (HDI).

Preferably, R″ is an unsubstituted C₄ alkylene (C₄H₈) or an unsubstituted C₃ alkylene (C₃H₆). R″ may be derived from a diol of the formula HO—R″—OH, such as 1,4-butanediol (BDO) or 1,3-propanediol (PDO).

Preferably, R″′ is an unsubstituted C₄ alkylene (C₄H₈) or an unsubstituted C₆ alkylene (C₆H₁₂). R′ may be derived from a diisocyanate of the formula O═C═N—R″′—N═C═O, such as alkanediisocyanate, preferably 1,4-butanediisocyanate (BDI) or 1,6-hexanediisocyanate (HDI).

A method for preparing phase-separated biodegradable polyurethanes of formula (I) is known in the art, such as for example described in WO-A-2004/062704.

An example of a polyurethane that can be very suitably used in the blend of the hemostatic foam of the invention is a phase separated polyurethane according to formula (I), wherein R is a polyether ester based on DL lactide and ε-caprolactone, which polyether ester comprises a hydrophilic polyethylene glycol segment; R′, R″ and R″′ are C₄ alkylene (C₄H₈); Q ¹, Q² and Z¹-Z⁴ are urethane and p=1 and q=1.

Another example that is preferred in accordance with the present invention is a structure wherein R=soft segment based on DL lactide and ε-caprolactone and polyvinylpyrrolidone as the hydrophilic segment.

Another example that is preferred in accordance with the present invention is a structure wherein R=soft segment based on DL lactide and ε-caprolactone and polyvinyl alcohol as hydrophilic segment.

For these latter two structures, R′, R″ and R″′ are C₄; Q¹, Q² and Z¹-Z⁴ are urethane; and p=1 and q=1.

The water-soluble polymer comprised in the polymer blend of the foam according to the present invention has a minimum molecular weight of preferably 300 g/mole, more preferably 600 g/mole. Furthermore, the water-soluble polymer comprised in the polymer blend of the foam according to the present invention has a maximum molecular weight of preferably 1,000,000 g/mole, more preferably 100,000 g/mole. For example, the water-soluble polymer comprised in the polymer blend of the foam according to the present invention may have a molecular weight of 20,000 g/mole.

A preferred example of a blend that may be used in the hemostatic foam of the invention is a blend of PEG and polyurethane, wherein said polyurethane comprises a soft PEG segment and a hard BDO-BDI-BDO segment.

A foam of the present invention has a density of 0.01-0.2 g/cm³, preferably of 0.03-0.07 g/cm³. Furthermore, a foam of the present invention has a porosity of 85-99%, preferably from 92-98%. A foam of the present invention has sufficient fluid absorption capacity at body temperature.

Foam according to the present invention may be “bioresorbable”. Bioresorbable refers to the ability of being completely metabolized by the human or animal body. This ability is suitable for certain applications, for example when a hemostatic agent is placed in an antrum or other body cavity.

The foam of the present invention is preferably for at least 99 wt. % based on the total weight of the foam synthetic, more preferably fully synthetic, thus comprising no materials derived from animals (e.g. collagen and gelatin) or plants (e.g. cellulose). During experiments in which a fully synthetic biodegradable foam according to the present invention was used for closure of an oroantral communication, appeared biocompatible with the tissue surrounding the implant. The term “biocompatible”, as used herein, refers to a material that upon contact with a living element of an organism, such as bleeding tissue, does not cause toxicity or injurious effects on the biological function of this tissue and organism. It is contemplated that the hemostatic foam of the invention may be used in-vivo without noticeable damage to organs, such as the kidney, upon use and subsequent biodegradation.

The hemostatic foam according to the present invention absorbs blood by its hydrophilic nature and porous structure and displays sufficient strength to remain properly positioned during the time of healing of the wound. New tissue may grow into the absorbent foam. After a certain period, which may be controlled by proper selection of the polyurethane used for its manufacture, the biodegradable hemostatic foam of the invention will degrade to mere residue and may eventually be completely metabolized by the body.

The hemostatic foam according to the invention can have any suitable shape, such as a cylinder, a cuboid, a plate, a flake or a cone. Some of these shapes are depicted in FIG. 2. Particular good results have been obtained using porous flakes, which may be porous irregular shaped particles with a size (largest diameter) varying from 0.5-4 mm. Porous flakes may be defined in the context of the present invention as containing sufficient pores (holes or other small cavities) so that the structure can hold a liquid or allow it to pass through. The porous flakes are advantageous in stopping excessive bleeding, because these flakes can easily be applied to surfaces which are normally difficult or even impossible to reach for a surgeon. Once the porous flakes are applied on the bleeding surface, coagulation will start and the flakes will stack together to form one large and dense blood clot. This allows for methods of treatment that were not possible before. If the porous flakes have different sizes and shapes, a better filling of the wound can be obtained, because a higher degree of packing can be obtained.

The hemostatic foam of the invention is suitable for stopping bleeding, in particular for stopping excessive bleeding. The hemostatic foam may be suitably used in applications such as teeth and molar extractions, surgeries in abdominal, ear, nose or throat (ENT), and general surgery, (partial) organ resection, tumor resection and craniotemie in cranio and maxillofacial surgery. The hemostatic foam of the invention is also suitable for packing antrums or other other cavities of the human or animal body, such as for example the nasal cavity. A further application of the hemostatic foam is use as an implant material, in particular for use in soft tissue repair.

In a further aspect, the present invention provides a method for preparing a hemostatic foam of the first aspect of the invention. This method comprises the following steps:
dissolving the polyurethane according to the first aspect in 1,4-dioxane or trioxane, more preferably 1,4-dioxane, thus obtaining a polyurethane solution
adding water-soluble polymer to said polyurethane solution
optionally adding cyclohexane
cooling down a mixture of said polyurethane solution and water soluble polymer to a temperature lower than 0° C., preferably lower than −18° C., obtaining a frozen mixture of water soluble polymer, polyurethane and either 1,4 dioxane or trioxane and cyclohexane if added.
allow at least part of said either 1,4 dioxane or trioxane and cyclohexane if added to sublime from the frozen mixture to the gas phase under reduced pressure, thus obtaining the hemostatic foam according to the present invention.

The present invention will be illustrated by the following examples, which is not intended to limit the scope of the present invention.

### EXAMPLES

### Example 1

### Comparative Study of Water Adsorption Speed

A first foam was prepared from a polyurethane based on a DL Lactide/ε-caprolactone soft amorphous segment including a PEG hydrophilic segment. The hard crystalline segment included 6 urethane groups (R′, R″ and R″′═C₄; Q¹, Q² and Z¹-Z⁴=urethane; and p=1 and q=1). A second foam was prepared from a polymer blend of the same polyurethane used for the first foam and polyethylene glycol. The ratio of polyurethane to polyethylene glycol in the polymer blend of the second foam was 3:1 by weight. The blood absorption capacity of the two foams was measured over time and the results are shown in FIG. 3. After one minute, the second foam had absorbed an amount of water from the blood eight times higher than the first foam. It was further observed that the second foam essentially retained its compression strength after absorption of the water.

### Example 2

### Comparative Study of Use as a Local Hemostatic Agent after Dental Extractions

The efficacy of polyurethane foam was assessed as a local hemostatic agent in sockets after dental extraction. The study design was a split mouth experiment, were three different hemostatic agents were compared.

Polyurethane (PU) foam had the polyester soft segments synthesized first, and consisted of (50/50) D/L lactide/ε-caprolactone and polyethylene glycol (PEG). Chain extension was performed resulting in polyurethane segments with a uniform length of 5 urethane moieties. The polyurethane polymer was dissolved in 1,4-dioxane. When the polymer was completely dissolved, PEGs was added. Cyclohexane was added when the PEG had completely dissolved. After cooling down the homogenous solution to −18 ° C., the frozen solution was freeze dried to remove the solvent crystals, obtaining a highly porous foam with a porosity of 93-97%. The ratio of polyurethane to polyethylene glycol in the polymer blend was 3:1 by weight. The PU foam was then cut into 1 cm pieces. The experimental samples used were of a cylindrical shape with an approximate diameter of 9 mm and an approximate height of 10 mm. The PU foam was packed in 4 cm blisters and sterilized using ethylene oxide.

The following agents were tested: PU foam according to the invention, Spongostan (absorbable gelatin sponge; Johnson & Johnson, Amersfoort, The Netherlands) and Hémocollagène (absorbable collagen haemostat; Septodont, Brussels, Belgium).

The study population consisted of 60 patients (>18 yrs) who required at least two extractions in one session. In the selected group of patients a standardized wound was available in duplicate so that different materials could be tested in individual patients. In each of the selected patients polyurethane foam was tested. In 30 patients the PU foam was compared to Spongostan and in another 30 patients the PU foam was compared to Hémocollagène.

Baseline measurements of blood were taken after molar extraction and before insertion of the foams. After the first extraction one of the foams will be placed in the socket. Only one kind of foam was tested in each socket. The foams remained in the socket for two minutes upon removal. After the removal, the foams were placed in a 1.5 ml micro tube with 250 μl 0.2 M EDTA and stored at approximately 4° C. The EDTA bound to the calcium in the blood, which stopped the thrombin formation. The second extraction followed the same procedure as mentioned above. When all the samples were obtained, the samples were centrifuged for 1 minute at 13,000 rpm using MSE Micro Centaur. After centrifuging, 200 μl of plasma was transferred to another 1.5 ml micro tube. The thrombin concentrations in the different samples were measured using ELISA (thrombin-antithrombin complex).

Besides thrombin concentration, the fibrinogen concentration in the different samples was also determined.

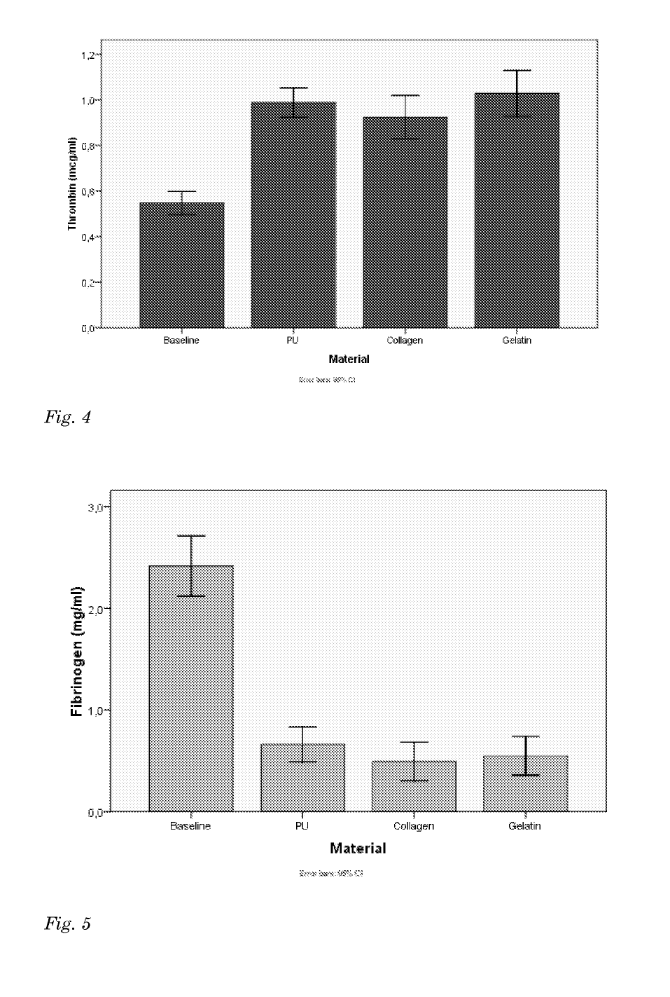

FIGS. 4 and 5 showed the thrombin and fibrinogen concentrations for the different samples. The PU foam of the invention had comparable results with the collagen and gelatin. All of the foams gave a significant increase of thrombin concentration and decrease of fibrinogen concentration in comparison to the baseline measurements.

### Example 3

### Tail-tip Test

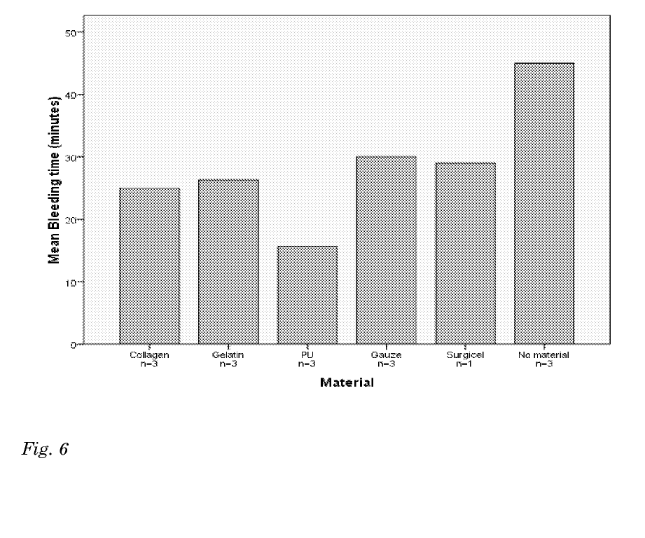

In this study comprising of 16 rats, the effectiveness of different materials as a hemostatic agent was assessed. The materials tested included the PU foam from Example 2, gelatin, collagen, Surgicel (oxidized cellulose) and gauze. The tail end was amputated 5 mm from the tip and the test materials were pressed to the end of the amputated tail. As a negative control, no material was used. The sample size for each of the test materials including the negative control was three, except for Surgicel where only one rat was used in the study. The time to hemostatis was then measured. FIG. 6 shows the hemostatic results of the different materials. It can be seen that the PU foam seems to generate coagulation the fastest of the materials tested.

1. Biodegradable hemostatic foam comprising a polymer blend of a water-soluble polymer and phase-separated polyurethane, comprising an amorphous segment and a crystalline segment, wherein at least said amorphous segment comprises a hydrophilic segment, wherein said polymer blend comprises from 0.5 to 20 times as much of said polyurethane as water-soluble polymer by weight.

2. Biodegradable hemostatic foam according to claim 1, wherein at least 50% of the atoms in the main chain of said water-soluble polymer is either part of a hydrophilic group or connected to a hydrophilic group through 2 atoms or less.

3. Biodegradable hemostatic foam according to claim 1, wherein said water-soluble polymer is a member of the group consisting of polyether, polypeptide, poly(vinyl alcohol), poly(vinyl pyrrolidone), poly(hydroxymethyl methacrylate) and copolymers thereof.

4. Biodegradable hemostatic foam according to claim 1, wherein said water-soluble polymer is polyethylene glycol.

5. Biodegradable hemostatic foam according to claim 1, wherein said polyurethane is of the formula:
R-Q¹[-R′-Z¹-[R″-Z²-R″′-Z³]ₚ-R″-Z⁴]_{q}-R′-Q²ₙ;
wherein R is selected from one or more aliphatic polyesters, polyetheresters, polyethers, polyanhydrides or polycarbonates, and at least one R comprises a hydrophilic segment; R′, R″ and R″′ are independently C₂-C₈ alkylene, optionally substituted with C₁-C₁₀ alkyl or C₁-C₁₀ alkyl groups substituted with protected S, N, P or O moieties or comprising S, N, P or O in the alkylene chain; Z¹-Z⁴ are independently amide, urea or urethane; Q¹ and Q² are independently urea, urethane, amide, carbonate, ester or anhydride; n is an integer from 5-500; and p and q are independently 0 or 1, provided that when q is 0, R is a mixture of at least one crystalline polyester, polyetherester or polyanhydride segment and at least one amorphous aliphatic polyester, polyether, polyanhydride or polycarbonate segment.

6. Foam according to claim 5, wherein Z¹-Z⁴ are urethane.

7. Foam according to claim 5, wherein p=1 and q=1.

8. Foam according to claim 1, wherein said polymer blend comprises from 2 to 5 times as much of said polyurethane as water-soluble polymer by weight.

9. Foam according to claim 1, wherein said foam is bioresorbable.

10. Foam according to claim 1, wherein said foam essentially retains its compression strength when saturated with blood.

11. Foam according to claim 1, wherein said foam is fully synthetic.

12. Porous flakes comprising a foam according to claim 1, which flakes have a size of 0.5-4 mm, and wherein said flakes are of different size and shapes.

13. Foam or flakes according to claim 1, for use in a method of treatment of a human or an animal.

14. Foam or flakes according to claim 13, where said treatment comprises controlling of bleeding, wound closure, prevention of tissue adhesion, support of tissue regeneration, or combinations thereof.

15. Process for the preparation of a foam according to claim 1, comprising at least the following steps:
dissolving the polyurethane according to the first aspect in either 1,4-dioxane or trioxane, thus obtaining a polyurethane solution
adding water-soluble polymer to said polyurethane solution
cooling down a mixture of said polyurethane solution and water-soluble polymer to a temperature lower than 0° C., obtaining a frozen mixture of water-soluble polymer, polyurethane and either 1,4 dioxane or trioxane.
allow at least part of said either 1,4 dioxane or trioxane to sublime from the frozen mixture to the gas phase under reduced pressure, thus obtaining the hemostatic foam according to the present invention.
